(19) United States
(12) Patent Application Publication
(10) Pub. No.: US 2021/0322216 A1
(43) Pub. Date: Oct. 21, 2021

(54) OPHTHALMIC SURGICAL INSTRUMENT AND METHOD OF USE

(71) Applicants: ART, LIMITED, Grand Cayman (KY); RAICO International LLC, Westmont, IL (US)

(72) Inventor: Takayuki AKAHOSHI, Tokyo (JP)

(21) Appl. No.: 17/266,495

(22) PCT Filed: Apr. 8, 2019
(86) PCT No.: PCT/US19/26330

§ 371 Date: Feb. 5, 2021

Related U.S. Application Data
(60) Provisional application No. 62/716,095, filed on Aug. 8, 2018.
(60) Provisional application No. 62/733,245, filed on Sep. 19, 2018.
(60) Provisional application No. 62/792,614, filed on Jan. 15, 2019.

(51) Int. Cl. A61F 9/007

(52) U.S. Cl. CPC A61F 9/00754; A61F 2230/0023

(57) ABSTRACT

An ophthalmic surgical instrument for nucleus splitting includes an instrument handle having proximal and distal ends, wherein the distal end includes a pair of cooperating blade mounts which can be selectively moved relative to each other by manipulation of the handle. A pair of blade elements are respectively joined to the blade mounts at the distal end of the handle. Each of the blade elements have a lower cutting surface positioned generally beneath an axis defined by the respective blade mount. Each of the blade elements has a non-cutting, blunt surface positioned generally above the axis of the respective blade mount. The lower cutting surfaces define a cutting edge for penetration of the nucleus of the eye when brought.

### FIELD OF THE INVENTION

The present invention relates generally to surgical instruments, and more particularly to ophthalmic surgical instruments having a pair of cooperating blade elements, which together define a cutting tip when placed in an abutting relationship, with the instruments being particularly configured to facilitate incision and splitting of the nucleus of a lens such as during a cataract removal procedure.

### BACKGROUND OF THE INVENTION

Phacoemulsification has come to be a technique of choice for the removal of damaged or diseased natural lenses from the eye. Commonly, such surgery is called for when a patient develops cataracts, a condition in which a portion of the eye lens becomes hard and opaque. Unless the damaged lens is removed and replaced with a properly selected artificial lens, blindness or severely impaired vision will result.

Phacoemulsification is the use of ultrasonic energy to emulsify the damaged lens and aspirate the resulting lens particles from the eye. One of the most significant advantages of the use of phacoemulsification is that the apparatus itself is small and can fit through a relatively small incision, resulting in less fluid leakage from the eye capsule and shorter patient recovery times.

It is desirable to limit the amount of ultrasonic energy used as much as possible in order to minimize the risk of damage to eye tissue. Often, the lens nucleus (the hardest portion of the lens) is chopped or split into smaller pieces prior to or during phacoemulsification. Smaller pieces require less energy to emulsify, and this shortens the time during which ultrasonic energy is actually being supplied to the phacoemulsification apparatus.

Known fractionating techniques include making incisions into the lens and, thereafter, prying the incisions open to split the lens into halves or quarters. U.S. Pat. No. 6,262,682, hereby incorporated by reference in its entirety, discloses a surgical instrument which facilitates the chopping and splitting of the lens nucleus.

The present invention is directed to an improved construction for an ophthalmic surgical instrument which facilitates chopping and splitting of the lens nucleus, and separation of the nucleus fragments.

### SUMMARY OF THE INVENTION

In accordance with the present invention, an ophthalmic surgical instrument for nucleus splitting separation is disclosed which is particularly configured to facilitate chopping and splitting of a lens nucleus, and separation of the broken pieces of the nucleus. The instrument includes a handle having proximal and distal ends, wherein the distal end includes a pair of cooperating blade mounts which can be selectively moved relative to each other by manipulation of the handle.

The instrument includes a pair of blade elements respectively joined to the blade mounts at the distal end of the instrument handle. Each of the blade elements has a lower cutting surface positioned generally beneath an axis that is defined by the respective blade mount of each blade element. Each of the blade elements has a non-cutting, blunt surface positioned generally above the axis of the respective blade mount. The lower cutting surfaces, when brought into a contacting relationship, define a cutting edge for penetration of the nucleus of the eye.

In another aspect of the present invention, the blunt surface has a first thickness and the lower cutting surface has a second thickness, both thicknesses in the direction normal to the central axis of the blade mount, and the second thickness is substantially less than the first thickness of the blunt surface.

In another aspect of the present invention, the blade elements have a distal end and a tapering configuration from their blunt surface to their lower cutting surface, when viewed looking toward the blade mount in a plane that is normal to the axis and taken through the blade element distal end.

In yet another aspect of the present invention, each of the blade elements has a distal end and each of the blade elements has a generally elliptical shape having a major axis and a minor axis. The major axis extends between the blade mount and the distal end. The minor axis extends between the blunt surface and the lower cutting surface and is perpendicular to the major axis.

In one aspect of the present invention, each of the blade elements has a distal end and each of the blade elements has a plurality of cutouts on its lower cutting surface to define at least one tooth or a plurality of teeth. The teeth preferably have a generally triangular shape.

In still another form of the present invention, the lower cutting surface of each of the blade elements is substantially straight. Preferably, the substantially straight lower cutting surface of each of said blade elements is substantially parallel to the axis defined by the respective blade mount of each blade element.

In another aspect of the present invention, the blunt surface of each of the blade elements is rounded.

In another aspect of the present invention, at least one of the blade elements has an indicium or indicia for indicating the location of one of either the blunt surface or the lower cutting surface.

In another broad form of the present invention, an ophthalmic surgical instrument for nucleus splitting separation is disclosed which is particularly configured to facilitate chopping and splitting of a lens nucleus, and separation of the broken pieces of the nucleus. The instrument includes a handle having proximal and distal ends, wherein the distal end includes a pair of cooperating blade mounts which can be selectively moved relative to each other by manipulation of the handle.

The instrument includes a pair of blade elements respectively joined to the blade mounts at the distal end of the instrument handle. Each of the blade elements has a lower cutting surface positioned generally beneath an axis that is defined by the respective blade mount of each blade element. Each of the blade elements has a non-cutting, blunt surface positioned generally above the axis of the respective blade mount. The lower cutting surfaces terminate at a pointed cutting tip for penetration of the nucleus.

The handle of the present surgical instrument can be configured generally as forceps to activate and manipulate the cooperating blade elements of the instrument by either a regular action, or a reverse action. That is, a hand grip portion of the instrument handle can be configured such that it can be squeezed to move the blade elements toward each other (regular action) or can be configured such that the hand grip portion of can be squeezed to move said blade elements away from each other (reverse action.)

The inventor has found that the surgical instruments according to the present invention are more suitable for improving safety and maneuverability during pre-chopping or chopping of the nucleus. Conventional, prior art pre-choppers have a narrow blade with a sharp edge that have been found to not provide sufficient separating force to the bisected nucleus. Furthermore, such prior art pre-choppers pose a danger to the delicate lens capsule with their sharp edge.

Other features and advantages will become readily apparent from the accompanying drawings and the appended claims.

### BRIEF DESCRIPTION OF THE DRAWINGS

In the accompanying drawings forming part of the specification, in which like numerals are employed to designate like parts throughout the same,

FIG. 1 is a greatly enlarged, fragmentary view of a portion of a first embodiment of an ophthalmic surgical instrument according to the present invention wherein only a fragmentary, distal portion of the instrument blade mounts and blades are illustrated in FIG. 1;

FIG. 2 is a front elevation view of the portion of the instrument shown in FIG. 1;

FIG. 3 is a perspective view, taken from above and to the right, of the portion of the instrument shown in FIG. 1, and FIG. 3 shows the instrument with the blades opened in a nucleus splitting position;

FIG. 4 is a is a top elevation view of the portion of the instrument shown in FIG. 1, and FIG. 4 shows the blunt top surface of each blade element;

FIG. 5 is a is a bottom elevation view of the portion of the instrument shown in FIG. 1, and FIG. 5 shows the lower cutting surface of each blade element closed in a nucleus chopping position;

FIG. 6 is a greatly enlarged, fragmentary view of a portion of a second embodiment of an ophthalmic surgical instrument according to the present invention wherein only a fragmentary, distal portion of the instrument blade mounts and blades are illustrated in FIG. 6;

FIG. 7 is a greatly enlarged, fragmentary view of a portion of a third embodiment of an ophthalmic surgical instrument according to the present invention wherein only a fragmentary, distal portion of the instrument blade mounts and blades are illustrated in FIG. 7;

FIG. 8 is a perspective view, taken from above and to the right, of the portion of the instrument shown in FIG. 7, and FIG. 8 shows the instrument with the blades opened in a nucleus splitting position;

FIG. 9 is a right-side elevation view of a fourth embodiment of an ophthalmic surgical instrument according to the present invention;

FIG. 10 is an enlarged, perspective view, taken from above and to the right, of a distal portion of the instrument shown in FIG. 9, and FIG. 10 shows the instrument with the blades opened in a nucleus splitting position;

FIG. 11 is a greatly enlarged, rotated, fragmentary, side view of the distal portion of the instrument shown in FIG. 10;

FIG. 12 is a right-side elevation view of a fifth embodiment of an ophthalmic surgical instrument according to the present invention;

FIG. 13 is an enlarged, fragmentary, perspective view, taken from above and to the right, of a distal portion of the instrument shown in FIG. 12;

FIG. 14 is a greatly enlarged, rotated, fragmentary, side view of the distal portion of the instrument shown in FIG. 12; and

FIGS. 15a-15g show a diagrammatic sequence of the operation of the first illustrated embodiment of an instrument according to the present invention, and FIGS. 15a-15g show the instrument chopping and splitting the nucleus of the human eye.

### DETAILED DESCRIPTION OF THE PREFERRED EMBODIMENTS

While the present invention is susceptible of embodiment in various forms, there is shown in the drawings and will hereinafter be described the presently preferred embodiments, with the understanding that the present disclosure should be considered as an exemplification of the invention and is not intended to limit the invention to the specific embodiments illustrated.

In accordance with the illustrated embodiments, the present ophthalmic surgical instrument 10 comprises an instrument handle 12 having proximal and distal ends, wherein the distal end includes a pair of cooperating blade mounts 14 which can be selectively moved relative to each other by manipulation of a hand grip portion of the handle 12 located intermediate of the proximal and distal ends.

As will be further described, the instrument further includes a pair of blade elements 16, which are a mirror image of one another, and are respectively joined to the blade mounts 14 at the distal end of the instrument handle 12. Each of the blade elements 16 includes a lower cutting surface 18, positioned generally beneath an axis 19 defined by the respective blade mount 14, and non-cutting, blunt surface 20 positioned generally above the axis 19 of the respective blade mount 14. Each of the blade elements 16, includes an exterior surface 22 and an interior surface 24 facing or opposing the adjacent blade element 16. The exterior surfaces 22 of each blade element 16 may be substantially straight between the cutting surfaces 18 and the blunt surface 20 or may be curved.

Notably, each of the cutting surfaces 18, when brought into a contacting, adjacent relationship, defines at least one, non-pointed or pointed, cutting surface or edge for penetration of the nucleus.

In the illustrated embodiments, it is contemplated that each blade element 16 has an outside dimension on the order of 2.2 mm.

The handle 12 of the present surgical instrument 10 can be configured generally as forceps to activate and manipulate the cooperating blade elements 16 of the instrument by either a regular action, or a reverse action. That is, the hand grip portion of the instrument handle 12 can be configured such that it can be squeezed to move the blade elements 16 toward each other (regular action) or can be configured such that the hand grip portion of can be squeezed to move said blade elements away from each other (reverse action.)

The blade elements of the illustrated shapes can also be designed on micro handles, for movement through a micro-incision (i.e., less than about 1.0 mm.)

The handle body of the instrument 10 can be made of lightweight titanium or medical grade stainless steel. The blade elements of the instrument can be made of stainless steel of various grades, including 420SS, 304SS, and 17-4 pH.

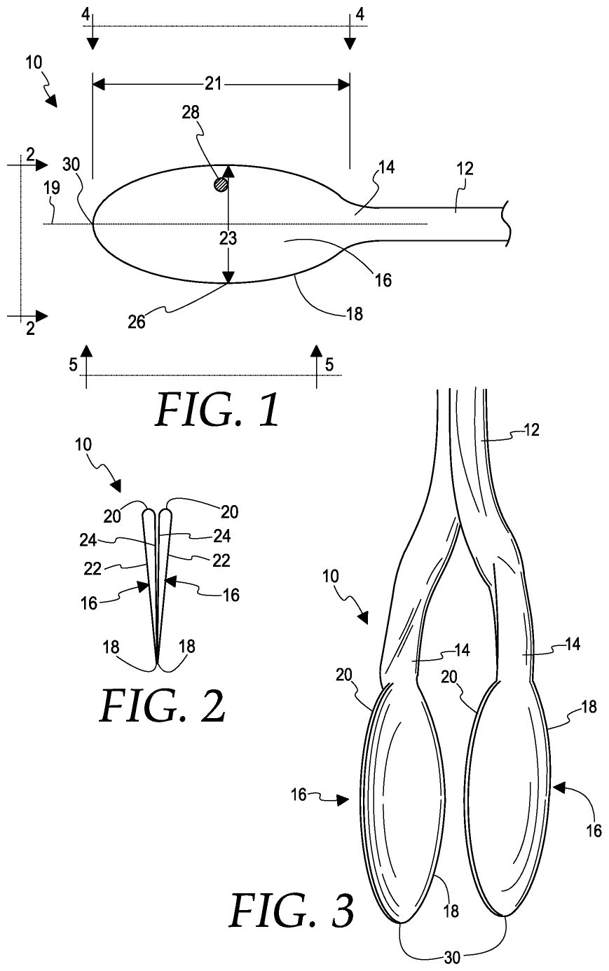

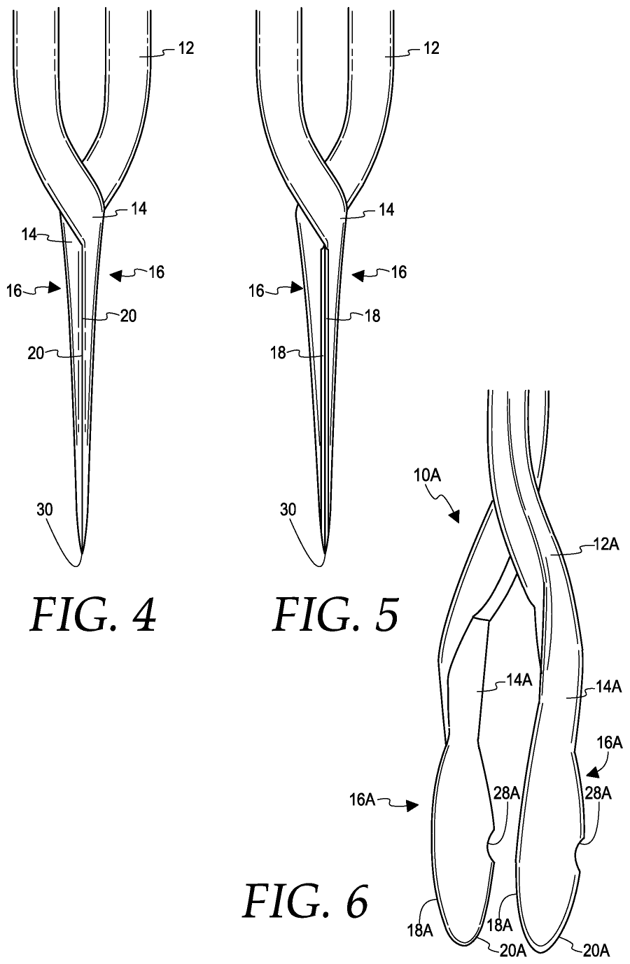

A first illustrated embodiment of a surgical instrument 10 according to the present invention is shown in FIGS. 1-5, wherein each of its blade elements 16 has a generally elliptical shape that defines a major axis 21 and a minor axis 23. The length of the major axis 21 is preferably greater than two times the length of the minor axis 23. Preferably, the major axis 21 extends in a collinear or coextensive manner with the axis 19 of the blade mount 14. However, it will be appreciated that the major axis 21 may be transverse or offset from the axis 19 of the blade mount 14 for some applications.

Referring to FIG. 1, the lower cutting surface 18 has a medial point 26, through which the minor axis 23 extends, the minor axis 23 being perpendicular to the major axis 21.

Still referring to FIG. 1, each lower cutting surface 18 extends from the blade mount 14 along one edge of the elliptical blade element 16 and terminates at a distal end 30 of the blade element 16, proximate to the axis 19. The blunt surface 20 extends along the opposite edge of the elliptical blade element 16 from the blade mount 14 to the distal end 30. When the blade elements 16 are brought together during operation by a surgeon, then the lower cutting surface 18 becomes a cutting edge in the form of an arc. Likewise, when the blade elements 16 are brought together during operation by a user, then the upper blunt surface 20 has the form of an arc.

With reference to FIG. 2, it can be seen that each blade element 16 has a greater thickness proximate the blunt surface 20 and a reduced thickness at the lower cutting surface 18.

The inventor has found that the first illustrated embodiment of the instrument 10, having elliptical/paddle shaped blade elements 16, is particularly advantageous and is most suitable for a soft nucleus and for a nucleus fragmented by a femtosecond laser.

A second embodiment of a surgical instrument according to the present invention is shown in FIG. 6, designated by the numeral 10A, and functions identically to the first illustrated embodiment of the instrument 10 as previously described. The numbered features of the second embodiment of the instrument 10A illustrated in FIG. 6 are designated generally with the suffix letter “A” and are analogous to features of the first embodiment of the instrument 10 that share the same number (without the suffix letter “A”). The second embodiment of the surgical instrument 10A differs from the aforementioned first illustrated embodiment of the instrument 10 in that the second embodiment includes indicia 28A in the form of a notch in each blade element 16A proximate to the blunt surface 20A. The indicia 28A enable the surgeon to quickly identify the location of the blunt surface 20A relative to the cutting surface 18A on each blade element 16A of the instrument 10A. The indicia 28A may have the form of a notch, recess, through hole, etching, coloring, or protrusion (not illustrated) on the exterior surface 22A of the blade element 16A. The indicium or indicia 28A may be omitted altogether.

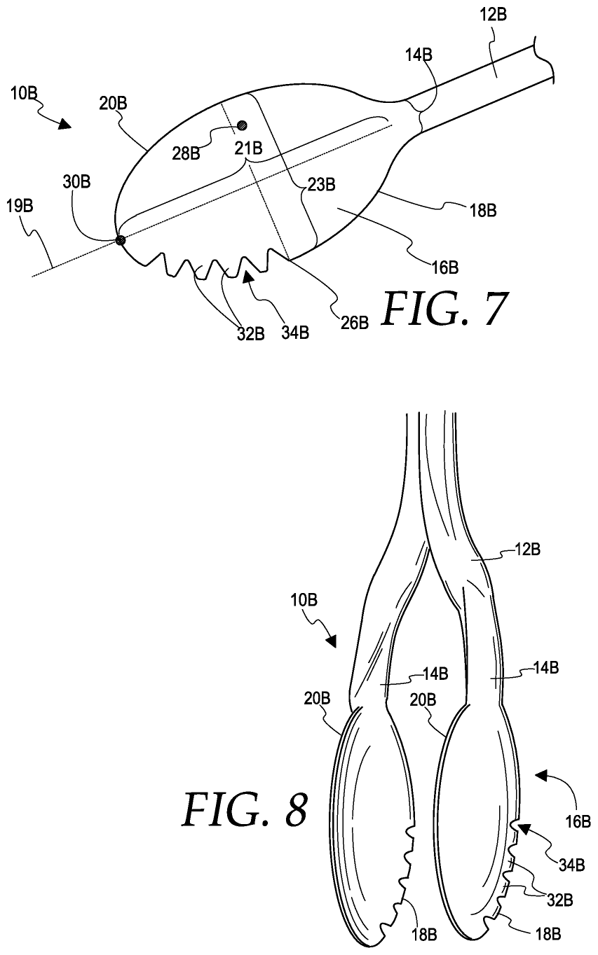

A third embodiment of a surgical instrument according to the present invention is shown in FIGS. 7 and 8, designated by the numeral 10B, and functions nearly identically to the first illustrated embodiment of the instrument 10 as previously described. The numbered features of the third embodiment of the instrument 10B illustrated in FIGS. 7 and 8 are designated generally with the suffix letter “B” and are analogous to features of the first embodiment of the instrument 10 that share the same number (without the suffix letter “B”). The instrument 10B has blade elements 16B which also have a generally elliptical shape that defines a major axis 21B and a minor axis 23B. The length of the major axis 21B is preferably greater than two times the length of the minor axis 23B. Preferably, the major axis 21B extends in a collinear or coextensive manner with the axis 19B of the blade mount 14B. However, it will be appreciated that the major axis 21B may be transverse or offset from the axis 19B of the blade mount 14B for some applications.

Referring to FIG. 7, the lower cutting surface 18B of each blade element 16B has a medial point 26B, through which the minor axis 23B extends, the minor axis 23B being perpendicular to the major axis 21B. Each lower cutting surface 18B extends from its blade mount 14B along one edge of the elliptical blade element 16B and terminates at a distal end 30B of the blade element 16B, proximate to the axis 19B. The lower cutting surface 18B differs from the prior-discussed embodiments in that it includes a plurality of teeth, serrations, or semi-sharp edges 32B separated by cutouts 34B. The teeth 32B generally extend only between the medial point 26B to the distal point 30B of each blade element 16B.

Still referring to FIG. 7, the blunt surface 20B extends along the opposite edge of the elliptical blade element 16B from the blade mount 14B to the distal end 30B. When the blade elements 16B are brought together during operation by a user, then the lower cutting surface 18B becomes a cutting edge in the form of a partially-serrated arc. When the blade elements 16B are brought together during operation by the user, then the upper blunt surface 20B has the form of an arc.

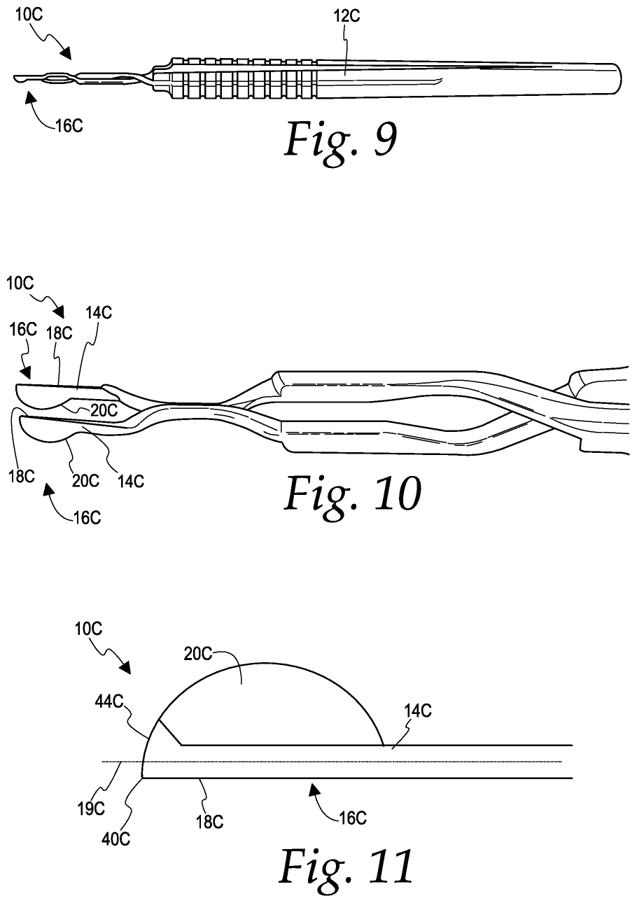

A fourth embodiment of a surgical instrument according to the present invention is shown in FIGS. 9-11, designated by the numeral 10C, and functions similarly to the first illustrated embodiment of the instrument 10 as previously described. The numbered features of the fourth embodiment of the instrument 10C illustrated in FIGS. 9-11 are designated generally with the suffix letter “C” and are analogous to features of the first embodiment of the instrument 10 that share the same number (without the suffix letter

With reference to FIG. 11, each blade element 16C of the fourth illustrated embodiment of the instrument 10C is substantially semi-circular (one being shown) and includes a lower, sharpened cutting surface 18C having a linear configuration, positioned beneath the axis 19C of the respective blade mount 14C. The lower cutting surface 18C of each blade element 16C terminates in a cutting tip 40C. In this embodiment of the instrument 10C, the tip portion of each blade element 16C is sharpened so that the cutting tip 40C extends generally from the lower, cutting surface 18C of each blade element 16C to a location above the axis 19C of the respective blade mount 14C.

In this embodiment of the instrument 10C, each blade element 16C includes a radiused, blunt surface 20C or edge located generally opposite the sharpened cutting surface 18C, with the blunt surface 20C positioned above the axis 19C of the respective blade mount 14C. The cutting tip 40C merges into the radiused blunt surface 20C of the blade element 16C in a radiused cutting surface 44C which extends across (above) the axis 19C.

The inventor has found that the fourth illustrated embodiment of the instrument 10C, having semi-circular shaped blade elements 16C which terminate in a cutting tip 40C at the distal ends, is especially suitable for a Grade 2+/3− nucleus, which may present a particularly difficult problem for prior art pre-chopping instruments. Such a nucleus is typically too hard to pre-chop using some prior art instruments, and yet such a nucleus is not hard enough for other prior art pre-chopping instruments.

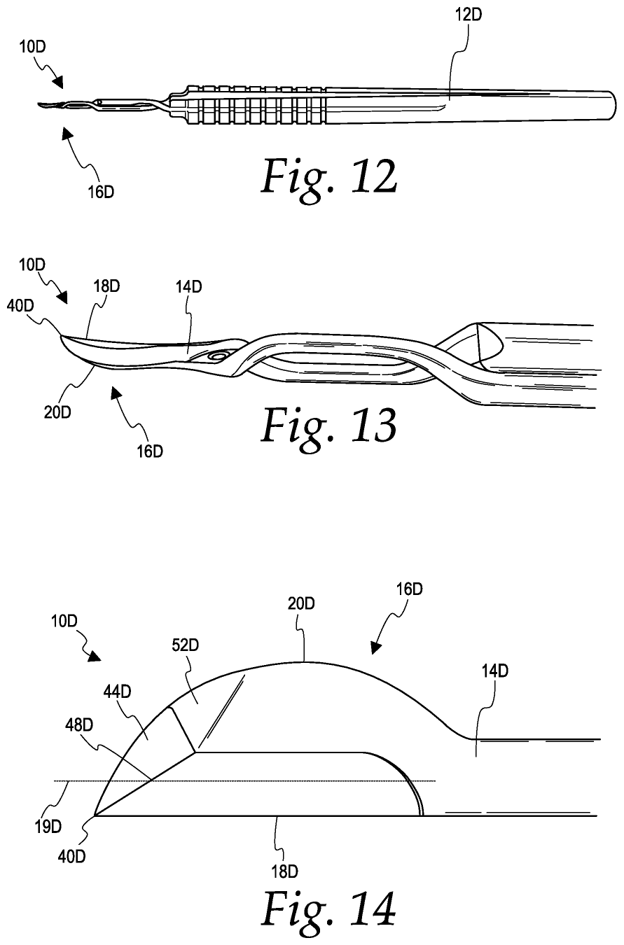

A fifth embodiment of a surgical instrument according to the present invention is shown in FIGS. 12-14, designated by the numeral 10D, and functions similarly to the fourth illustrated embodiment of the instrument 10C as previously described. The numbered features of the fifth embodiment of the instrument 10D illustrated in FIGS. 12-14 are designated generally with the suffix letter “D” and are analogous to features of the fourth embodiment of the instrument 10C that share the same number (without the suffix letter “C”).

Referring now to FIG. 14, each blade element 16D includes a lower, sharpened cutting surface 18D having a linear configuration, positioned generally beneath the axis 19D of the respective blade mount 14D. Each blade includes a radiused blunt surface 20D positioned above axis 19D of the respective blade mount 14D. The lower cutting surface 18D is sharpened to extend in a direction generally away from axis 19D.

Still referring to FIG. 14, the lower cutting surface 18D of each blade element 16D defines and terminates in a cutting tip 40D. In this embodiment of the instrument 10D, the cutting tip 40D is defined and formed at an intersection between: (i) the lower cutting surface 18D; and (ii) a radiused cutting surface 44D which is sharpened to extend in a direction generally away from lower cutting surface 18D. A raised ridge or region of greater blade thickness 48D forms a boundary between the lower cutting surface 18D and the radiused cutting surface 44D.

As illustrated FIG. 14, the radiused cutting surface 44D is generally defined by a right angle, with the radiused cutting surface 44D merging into the blunt edge 20D at a radiused blunt edge portion 52D of each blade element.

In effect, the embodiment of the instrument 10D illustrated in FIGS. 12-14 provides a “double-edged” cutting instrument, including the lower cutting surface 18D, and the radiused cutting surface 44D. It is contemplated that the resultant V-pointed cutting tip 40D facilitates penetration of the nucleus, particularly in in the instances of a more mature or harder cataract. The relatively thin profile of the cutting blades 16D also facilitates penetration of the nucleus, while the radiused blunt edge 20D of each cutting blade 16D can be used to rotate and separate the nucleus once it is cracked.

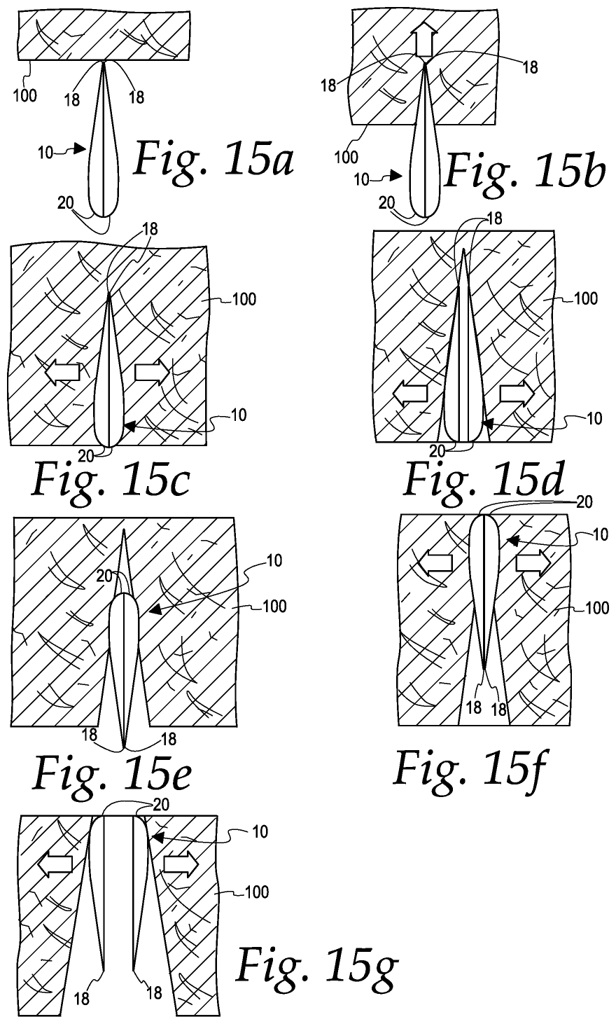

FIGS. 15a-e illustrates the general operation of any one of the surgical instruments 10, 10A, 10B, 10C, 10D according to the present invention, however only surgical instrument 10 is illustrated in FIG. 15. The surgical instruments 10, 10A, 10B, 10C, and 10D have been found by the inventor to have an improved performance over one or more pre-choppers of the prior art in one or more of the following categories: insertion into the eye, separation of the nucleus, rotation and maneuverability of the instrument within the eye, and safety when separating the nucleus

From the foregoing, it will be observed that numerous modifications and variations can be effected without departing from the true spirit and scope of the novel concept of the present invention. It is to be understood that no limitation with respect to the specific embodiments illustrated herein is intended or should be inferred. The disclosure is intended to cover, by the appended claims, all such modifications as fall within the scope of the claims.

1. An ophthalmic surgical instrument (10, 10A, 10B, 10C, 10D) for nucleus splitting, comprising:
an instrument handle (12, 12A, 12B, 12C, 12D) having proximal and distal ends, wherein the distal end includes a pair of cooperating blade mounts (14, 14A, 14B, 14C, 14D) which can be selectively moved relative to each other, and
a pair of blade elements (16, 16A, 16B, 16C, 16D) respectively joined to said blade mounts (14, 14A, 14B, 14C, 14D) at the distal end of said instrument handle (12, 12A, 12B, 12C, 12D),
each of said blade elements (16, 16A, 16B, 16C, 16D) having a lower cutting surface (18, 18A, 18B, 18C, 18D) positioned generally beneath an axis (19, 19A, 19B, 19C, 19D) defined by the respective blade mount (14, 14A, 14B, 14C, 14D),
each of said blade elements (16, 16A, 16B, 16C, 16D) having a non-cutting, blunt surface (20, 20A, 20B, 20C, 20D) positioned generally above the axis (19, 19A, 19B, 19C, 19D) of the respective blade mount (14, 14A, 14B, 14C, 14D), and
wherein said lower cutting surfaces (18, 18A, 18B, 18C, 18D) in a contacting relationship define a cutting edge for penetration of the nucleus.

2. The ophthalmic surgical instrument (10, 10A, 10B, 10C, 10D) for nucleus splitting in accordance with claim 1, wherein said blunt surface (20, 20A, 20B, 20C, 20D) has a first thickness and said lower cutting surface (18, 18A, 18B, 18C, 18D) has a second thickness that is substantially less than said first thickness of said blunt surface (20, 20A, 20B, 20C, 20D).

3. The ophthalmic surgical instrument (10, 10A, 10B) for nucleus splitting in accordance with claim 1, wherein each of said blade elements (16, 16A, 16B) has a distal end (30, 30A, 30B), and each of said blade elements (16, 16A, 16B) has a tapering configuration from said blunt surface (20, 20A, 20B) to said lower cutting surface (18, 18A, 18B) when viewed looking toward said blade mount (14, 14A, 14B) in a plane that is normal to said axis (19, 19A, 19B) and taken through said distal end (30, 30A, 30B).

4. The ophthalmic surgical instrument (10, 10A, 10B) for nucleus splitting in accordance with claim 1, wherein each of said blade elements (16, 16A, 16B) has a distal end (30, 30A, 30B) and a generally elliptical shape defining a major axis (21, 21A, 21B) and defining a minor axis (23, 23A, 23B), said major axis (21, 21A, 21B) extending between said blade mount (14, 14A, 14B) and said distal end (30, 30A, 30B), and said minor axis (23, 23A, 23B) extending between said blunt surface (20, 20A, 20B) and said lower cutting surface (18, 18A, 18B) and being perpendicular to said major axis (21, 21A, 21B).

5. The ophthalmic surgical instrument (10B) for nucleus splitting in accordance with claim 1, wherein each of said blade elements (16B) has a distal end (30B), and wherein each of said blade elements (16B) has a plurality of cutouts (34B) on said lower cutting surface (18B) to define at least one tooth (32B).

6. The ophthalmic surgical instrument (10B) for nucleus splitting in accordance with claim 5, wherein said lower cutting surface (18B) has a plurality of teeth (32B) extending from a medial point (26B) on said lower cutting surface (18B) to said distal end (30B) of each of said blade elements (16B).

7. The ophthalmic surgical instrument (10B) for nucleus splitting in accordance with claim 5, wherein said at least one tooth (32B) has a generally triangular shape.

8. The ophthalmic surgical instrument (10C, 10D) for nucleus splitting in accordance with claim 1, wherein said lower cutting surface (18C, 18D) of each of said blade elements (16C, 16D) is substantially straight.

9. The ophthalmic surgical instrument (10C, 10D) for nucleus splitting in accordance with claim 8, wherein said lower cutting surface (18C, 18D) of each of said blade elements (16C, 16D) is substantially parallel to said axis (19C, 19D) defined by the respective blade mount (14C, 14D).

10. The ophthalmic surgical instrument (10, 10A, 10B, 10C, 10D) for nucleus splitting in accordance with claim 1, wherein said blunt surface (20, 20A, 20B, 20C, 20D) of each of said blade elements (16, 16A, 16B, 16C, 16D) is rounded.

11. The ophthalmic surgical instrument (10C, 10D) for nucleus splitting in accordance with claim 1, wherein said lower cutting surface (18C, 18D) of each of said blade elements (16C, 16D) terminates at a pointed cutting tip (40C, 40D) for penetration of the nucleus.

12. The ophthalmic surgical instrument (10C, 10D) for nucleus splitting in accordance with claim 11, wherein each said pointed cutting tip (40C, 40D) includes a radiused cutting surface (44C, 44D) that extends from said lower cutting surface (18C, 18D) across said axis (19C, 19D) and merges into said blunt surface (20C, 20D).

13. The ophthalmic surgical instrument (10D) for nucleus splitting in accordance with claim 12, wherein each blade element (16D) further comprises a raised ridge (48D) that extends transversely with respect to the axis (19D) of the respective blade mount (14D) that separates said radiused cutting surface (44D) and said lower cutting surface (18D).

14. The ophthalmic surgical instrument (10D) for nucleus splitting in accordance with claim 13, wherein each blade element (16D) further comprises a radiused blunt edge portion (52D) located between said radiused cutting surface (44D) and said blunt surface (20D).

15. The ophthalmic surgical instrument (10C, 10D) for nucleus splitting in accordance with claim 11, wherein each said lower cutting surface (18C, 18D) extends a greater distance along each said axis (19C, 19D) than each said radiused cutting surface (44C, 44D) of each said blade element (16C, 16D).

16. The ophthalmic surgical instrument (10D) for nucleus splitting in accordance with claim 13, wherein each said ridge (48D) has (i) a maximum thickness proximate to a point of intersection with said axis (19D) and (ii) a decreasing thickness toward each said cutting tip (40D).

17. The ophthalmic surgical instrument (10, 10A, 10B, 10C, 10D) for nucleus splitting in accordance with claim 1, wherein at least one of said blade elements (16, 16A, 16B, 16C, 16D) has an indicium (28A) for indicating the location of one of said blunt surface (20, 20A, 20B, 20C, 20D) or said lower cutting surface (18, 18A, 18B, 18C, 18D).

18. An ophthalmic surgical instrument (10, 10A, 10B, 10C, 10D) for nucleus splitting in accordance with claim 1, wherein:
said instrument handle (12, 12A, 12B, 12C, 12D) can be squeezed to move said blade elements (16, 16A, 16B, 16C, 16D) together.

19. An ophthalmic surgical instrument (10, 10A, 10B, 10C, 10D) for nucleus splitting in accordance with claim 1, wherein:
said instrument handle (12, 12A, 12B, 12C, 12D) can be squeezed to move said blade elements (16, 16A, 16B, 16C, 16D) apart.
